(19) United States
(12) Patent Application Publication
(10) Pub. No.: US 2015/0362481 A1
(43) Pub. Date: Dec. 17, 2015

(54) METHOD FOR THE IDENTIFICATION OF BITTER TASTING COMPOUNDS AND BITTER TASTE MODULATING COMPOUNDS

(71) Applicant: Universitat Wien, Wien (AT)

(72) Inventors: Veronika Somoza, Weidling (AT); Kathrin Ingrid Liszt, Alservorstadt (AT); Elke Köck, Alservorstadt (AT); Jakob Ley, Holzminden (DE)

(21) Appl. No.: 14/761,804

(22) PCT Filed: Jan. 17, 2014
(86) PCT No.: PCT/EP2014/050950

§ 371 Date: Jul. 17, 2015

(30) Foreign Application Priority Data
Jan. 17, 2013 (EP) 13151578.5
Feb. 18, 2013 (EP) 13155658.1

(51) Int. Cl. G01N 33/50; G01N 21/64; G01N 33/84

(52) U.S. Cl. CPC G01N 33/5044; G01N 33/84; G01N 21/6428; G01N 2021/6439; G01N 2021/6497; G01N 2458/30

(57) ABSTRACT

The present invention is directed to a method for the identification of bitter tasting compounds and bitter taste modulating compounds, in particular bitter taste masking compounds (bitter antagonists) and bitter taste enhancing compounds (bitter agonists) by monitoring the change in the intracellular pH value and/or proton secretion.

### FIELD OF THE INVENTION

The present invention is directed to a method for the identification of bitter tasting compounds and bitter taste modulating compounds, in particular bitter taste masking compounds (bitter antagonists) and bitter taste enhancing compounds (bitter agonists) by monitoring the change in the intracellular pH value and/or proton secretion.

### STATE OF THE ART

Food and consumer products often contain a plurality of bitter tasting compounds (bitter agonists) which on the one hand are desirable in certain food products and which contribute to their characteristic taste (for example caffeine in tea or coffee, quinine in so called bitter-lemon beverages, bitter tasting compounds such as humulones or iso-α-acids derived from hop in beer) which, on the other hand, might lower their value as well. This group comprises flavanoid glycosides and limonoids in citrus juices, the bitter aftertaste of several high-intensity sweeteners such as aspartame, cyclamate, acesulfame K, rebaudioside A, glycyrrhizin or saccharine and the unpleasant taste which is caused by hydrophobic amino acids and peptides in cheese.

Bitter taste usually is caused by single compounds, which are binding to specific bitter receptors on taste cells located in the so-called taste buds on the tongue and which are transmitting a signal to the brain via neuro-chemical cascades, which in turn produces a defense reaction and a negative taste perception (see Meyerhof, Reviews of Physiology, Biochemistry and Pharmacology, 2005, 154, 37-72).

Therefore, it is desirable to find bitter taste masking compounds which effectively modify, decrease or even suppress unpleasant taste perceptions, in particular bitter, astringent and/or metallic taste perceptions, without influencing the quality of the respective food product or a corresponding preparation suitable for consumption. Bitter taste masking compounds are defined as molecules, which may directly influence the perception of bitter taste on a physiological level; not included in this definition are e.g. complex forming compounds such as cyclodextrine or ion-exchange resins, which are simply lowering the effective concentration of bitter tasting compounds, which have a pleasant taste, for example, salty, sweet or umami taste, thereby indirectly masking the bitter taste.

Those substances and principles are often used in the pharmaceutical area, since many active pharmaceutical ingredients taste bitter and thus are aversive to patients. Encapsulation of the medicine in different forms thus is used in practice, but often problematic for children since they cannot or will not swallow solid dose forms. Liquid formulations are often combined with more pleasant tasting compounds, for example, sucrose, high-intensity sweeteners, and flavors. Adding both sugars and acids to pharmaceutical formulations may reduce the bitterness of drugs. However, frequent use of sucrose-sweetened or acidic medicines has been linked to dental caries in children.

Thus, alternative approaches are needed to overcome the unpleasant taste of bitter substances, such as the development or identification of bitter taste masking substances.

Usually, bitter taste masking compounds are identified by the following processes:
by sensory methods, i.e. by comparing the taste of a mixture of the bitter tasting compound and the bitter antagonist with the taste of the bitter tasting compound alone;
by screening in presence of a bitter tasting compound with/without bitter antagonist by means of a heterologously expressed bitter receptor and accessory molecules if needed on immortalized animal, preferably human cells (see for example WO 2004/029087 A1, WO 2008/057470A1);
by screening in presence of a bitter tasting compound with/without bitter antagonist with native human or immortalized human taste cells; or
by means of a computer based prediction model by using pharmacophore models or homology models of the corresponding receptors.

Sensory screening is time- and work-consuming and can only be performed with toxicologically harmless substances. With the heterologously expressed systems, the correlation to the real sensory perception quite often can be displayed insufficiently only and, furthermore, for simplification only one receptor is expressed; since for the determination of the activation a calcium-sensitive fluorescent dye is added, interferences are common, in particular at high concentrations of the test substances. Primary or immortalized taste cells as a rule are difficult to cultivate and to stably obtain for a certain taste- or bitter level, furthermore, the receptor composition is not always constant. The calculation methods quite often are unspecific, since up to now no experimental three-dimensional structural data important for the homology calculations, for example X-ray crystal structure, are present for the taste receptors.

WO 03/031604 is related to STC-1 enteroendocrine cells expressing multiple bitter taste receptors which respond to bitter tasting compounds initiating changes in intracellular calcium concentrations (see the abstract). Example 5 demonstrates that STC-1 cells show rapid Ca²⁺ responses after contact with bitter tasting substances. Further, WO 03/031604 also indicates that taste receptor families identified in taste cells of the lingual epithelium are also expressed in the gastric (and duodenal) mucosa.

The complex problem underlying the present invention is, therefore, to develop a screening method for the specific identification of bitter taste antagonists, which, at the same time
(i) is based on a human cell culture suitable for high-throughput screening,
(ii) does not require heterologously expressed bitter receptors,
(iii) usually is not based on a direct measurement of the change in the calcium concentrations in the cell,
(iv) has a high correlation with the human taste perception,
(v) identifies both, bitter tasting compounds and bitter taste modulating compounds, i.e. bitter taste masking and bitter taste enhancing compounds, and in particular identifies bitter taste masking compounds which avoid the addition of additives or the use of formulation principles with potentially adverse effects on the patients.

### DESCRIPTION OF THE INVENTION

The present invention is directed to an in vitro method for the identification of bitter tasting compounds and bitter taste modulating compounds, where the test substances are brought into contact with isolated gastric cells or gastric tumor cells and where the resulting change in the intracellular pH value and/or cellular proton secretion is determined. Those test substances causing a change in the intracellular pH value and/or cellular proton secretion thus will be identified as bitter tasting compounds and bitter taste modulating compounds.

The gastric cells or gastric tumor cells preferably are proton secreting cells.

Surprisingly, it turned out that in particular cultivated gastric cells show a very good correlation between the sensory characteristics and in vitro data based on known test substances (bitter taste masking and non-effective test substances) via the change in the intracellular pH value and the proton secretion respectively, and, therefore, the system is quite suitable to screen a plurality of test compounds for identification of bitter antagonists of the above-mentioned bitter tasting compounds.

### Cell Systems

It turned out that preferred cell systems are isolated, proton secreting primary gastric cells or gastric tumor cell lines. Furthermore, it has been shown that in particular HGT-1 cells express genes of bitter receptors. An advantage is in particular the concomitant expression of several bitter receptors which has been proven as being advantageous for the subsequent comparative sensory measurement. Apart from HGT-1 cells, also alternative cell systems are considered herein, for example isolated gastric cells of the Provenienz rat (isolated rat gastric mucosal cells; Dixit, C.; Dikshit, M., A flow cytometric method for evaluation of acid secretion from isolated rat gastric mucosal cells. J PharmacolToxicol Methods 2001, 45, 47-53), rabbit (isolated gastric gland from white rabbits; Matsuno, K.; Tomita, K.; Okabe, S., Wine stimulates gastric acid secretion in isolated rabbit gastric glands via two different pathways. Aliment PharmacolTher 2002, 16 Suppl 2, 107-14), mouse (Vila-Petroff, M.; Mundiña-Weilenmann, C.; Lezcano, N.; Snabaitis, A. K.; Huergo, M. A.; Valverde, C. A.; Avkiran, M.; Mattiazzi, A., Ca⁽²⁺⁾/calmodulin-dependent protein kinase II contributes to intracellular pH recovery from acidosis via Na⁽⁺⁾/H⁽⁺⁾ exchanger activation. J Mol Cell Cardiol 2010, 49, 106-12) or guinea pig (Swietach, P.; Rossini, A.; Spitzer, K. W.; Vaughan-Jones, R. D., H⁺ ion activation and inactivation of the ventricular gap junction: a basis for spatial regulation of intracellular pH. Circ Res 2007, 100, 1045-54). Further suitable are gastric tumor cell lines such as MKN-45 (Nagata H, Che X F, Miyazawa K, Tomoda A, Konishi M, Ubukata H, Tabuchi T., Oncol Rep. 2011, 25341-6), AGS (Smolka A J, Goldenring J R, Gupta S, Hammond C E, Inhibition of gastric H,K-ATPase activity and gastric epithelial cell, BMC Gastroenterol. 2004 10:4-8.).

Up to now, gastric tumor cells such as HGT-1 cells have been used as a measurement system for the identification and characterization of substances, which might influence the gastric juice secretion, for example of certain sour tasting fruit acids (see Liszt, et al., J. Agric. Food Chem. 60, (28), 7022-7030 (2012). HGT-1 cells are well-known in the art and are described in detail, for example, in Laboisse C L, Augeron C, Couturier-Turpin M H, Gespach C, Cheret A M, Potet F., Cancer Res. 1982 April; 42(4):1541-8.

Furthermore, it is known that selected bitter tasting compounds such as hop ingredients (see Walker et al. J. Agric. Food Chem. 60, (6), 1405-1412 (2012) or ingredients of coffee (see Rubach et al., J. Agric. Food Chem. 58, 4153-61 (2010)) influence secretion, however, a general correlation between the sensory characteristic “bitter” and the physiological reaction “acid secretion” has not been established so far. Therefore, it was surprising that the HGT-1 cells are suitable as a measurement system for the identification and characterization of bitter agonists, thus, forming a further aspect of the invention.

Up to now, methods of identifying compounds having bitter taste relied on the change in intracellular calcium levels, see WO 03/031604 discussed above. However, it is shown herein for the first time that a correlation exists between the perception of bitter taste and proton secretion in gastric cells/gastric tumor cells. Therefore, bitter tasting substances or their antagonists may be identified by determining the proton secretion of these cells and/or the measurement of the intracellular pH. It is noted that the relation between the proton secretion and the intracellular pH is the following, i.e. the more protons (acid) are secreted by the gastric cells/gastric tumor cells used in the present method, the higher the intracellular pH of said cells will be. However, both values may be used as a read-out in the present method.

Furthermore, the only target for the reduction of secretion on the cell surface of HGT-1 cells (human gastric tumor cell line) has been described as somatostatin receptor (SSTR2), histamine receptor (HRH2) and acetylcholine receptor (CHRM3). Up to now, these cells have been brought into contact as single compounds with potential regulators of the proton secretion only, whereas the common administration of bitter agonists and potential bitter taste modulators, in particular bitter taste masking compounds, has not been described so far. Although taste receptors have been described in a plurality of non-oral tissues (see Behrens, et al., Physiology & Behavior 105, (1), 4-13 (2011)), no evidence has been given so far except for the oral cavity, that the activation of these receptors will lead to a perceptible sensory or taste event.

Therefore, it was surprising and unexpected for a skilled person that isolated gastric cells or gastric tumor cells, in particular HGT-1 cells, may be suitable for use in a method for the identification of taste modulating, in particular bitter taste modulating compounds.

By the method of the present invention, preferably bitter taste masking compounds may be identified. To put it simply, the development of the pH value is compared, i.e. the release of protons in two similar cell cultures, where to one sample only the known bitter tasting compound is added and to the other sample the known bitter tasting compound together with the one or more test substances is added. The amount of released protons is measured and the difference of the T/C value (treatment over control) is formed. If the following inequation applies:

T/C(known bitter tasting compound)−T/C(known bitter tasting compound+test substance)>0

i.e. if the difference is positive (value of the control=0), this will mean that in the sample which contains the test substance, fewer protons have been released. If the deviation from 0 is significant, then the substance is a potential bitter masking compound which will be further evaluated in additional sensory analysis. The deviation is significant, if the relative difference between both values is at least 10%, preferably at least 20% and in particular at least 30% of the higher value.

The ratio T/C expresses the proton release in treated (T) vs. untreated cells (C). The results so received then will be indicated as percent change compared to the untreated control cells. It is noted that “untreated” as referred to herein means that the gastric cells/gastric tumor cells are not treated with known bitter tasting compounds and/or test substances. However, the other conditions are the same than for treated cells, i.e. use of solvents, temperature conditions etc.

The identification of bitter taste masking compounds is one of the preferred aspects of the present invention. Surprisingly, gastric cells/gastric tumor cells may be used to identify those substances which mask the taste of other bitter tasting substances. See in particular the experimental results contained in Example 3, i.e. Tables 3A-3H showing the percent increase of caffeine/theobromine alone or in combination with the bitter-masking compounds.

The same process in turn can be used in order to identify bitter taste enhancing compounds. Here the inequation is:

T/C(known bitter tasting compound)−T/C(known bitter tasting compound+test substance)<0

i.e. the difference is negative (value of the control=0). This means that in the sample which contains the test substance, more protons have been released. Also in this case, the deviation is significant whenever the relative difference between both values is at least 10%, preferably at least 20% and in particular at least 30% of the higher value.

The method further is suitable for the identification of potential bitter tasting compounds. In this case, the inequation is as follows:

T/C(control)−T/C(test substance)<0

where T/C is the value of the proton secretion in percent under the condition that the relative difference of the two values [the value of the neutral control (solvent and buffer without test substance) and the value of the test substance] is at least 10% of the higher value. In this specific case, the test substance is added without agonist and it will be determined, whether a significant increase of proton secretion takes place.

A further embodiment of the present invention is directed to a method for identifying the degree of the bitter taste of a bitter tasting compound. The method is performed as described above, but the results are additionally compared with test results obtained for one or more negative or positive control substances. The bitter taste of the bitter tasting compound relative to these control substances then may be determined.

A further aspect of the present invention is directed to a method for the identification of bitter taste modulating compounds of the above described type, where,
(a) a uniform culture of a cell system is provided selected from the group comprising isolated gastric cells or gastric tumor cells which is divided in two samples,
(b) to the first sample, a known bitter tasting compound (bitter agonist) is added,
(c) to the second sample, the same bitter tasting compound of step (b) and at least one test substance is added, which test substance can be one or more bitter taste modulating (i.e. bitter taste enhancing or bitter taste masking) compound,
(d) measuring the change in the intracellular pH value and/or the proton secretion of the cells during the course of the experiment,
(e) after completing the experiment, the difference between the change in the intracellular pH value and/or the proton secretion of the cells of the first and the second sample is calculated,
(f) those test substances are selected, where the difference is positive or negative and the relative difference between both values is at least 10% of the higher value, and
(g) optionally, subjecting the so identified test substances to a sensory evaluation.

In a preferred embodiment of the invention, the gastric cells or gastric tumor cells are proton secreting cells.

Additionally, a method for the identification of bitter tasting compounds of the above described type is claimed where:
(a) a uniform culture of a cell system is provided selected from the group comprising isolated gastric cells or gastric tumor cell lines, which is divided in two samples,
(b) to the first sample, a control solution without test substance is added,
(c) to the second sample, a test substance is added,
(d) the change in the intracellular pH value and/or proton secretion of the cells is measured during the course of the experiment,
(e) after completing the experiment, the difference between the change in the intracellular pH value and/or proton secretion of the cells of the first and the second sample is calculated,
(f) those test substances are selected where the difference is negative and where the relative difference between both values is at least 10% of the higher value,
(g) and, optionally, the so identified test substances subsequently are subjected to a sensory evaluation for the determination of the bitter effect.

In a still further aspect, the present invention is directed to a kit for performing the method described herein. The kit is adapted to the screening for bitter tasting or bitter taste modulating substances. Such kits can be prepared from readily available materials and reagents. For example, such kits can comprise any one or more of the following materials: a suitable media containing gastric cells/gastric tumor cells, reaction tubes or the like suitable devices, and instructions for performing the method. Depending on the precise use of the kit, for example identification of bitter taste masking and bitter taste enhancing compounds, the kit may further comprise one or more known bitter tasting compounds. Further ingredients of the kit may be means needed for measuring the intracellular pH value and/or the proton secretion of the cells such as a fluorescent dye for spectrometric measurement.

A wide variety of kits and components can be prepared according to the present invention, depending upon the intended user of the kit and the particular needs of the user.

### Known Bitter Tasting Compounds (Bitter Agonists)

Known bitter tasting compounds (bitter agonists) which might be added to the cell cultures according to the present invention, are compounds which, alone and in a suitable amount, may trigger a bitter taste in aqueous solution systems in a human being and which may activate one or more of the known 25 human bitter receptors, in particular receptors of type TAS2R1, TAS2R3, TAS2R7, TAS2R10, TAS2R14, TAS2R16, TAS2R20, TAS2R30, TAS2R38, TAS2R40, TAS2R43, TAS2R46 and TAS2R50 which have already been detected in HGT-1 cells.

Preferably, the bitter agonists are selected from the group of:
xanthine alkaloids, e.g. caffeine, theobromine, theophylline;
alkaloids, e.g. quinine, brucine, strychnine, nicotine;
phenolic glycosides, e.g. salicin, sinigrin, arbutin;
flavonoid glycosides, e.g. neohesperidin, eriocitron, neoeriocitron, nairutin, naringin;
chalconesor chalcone glycosides, dihydrochalconegylocides, e.g. phloridzin, trilobatin;
hydrolysable tannins, e.g. gallic or ellagic acid esters of carbohydrates, e.g. pentagalloyl glucose;
non-hydrolysable tannins, e.g. galloylisedcatechins or epicatechins and oligomers thereof, e.g. proanthyocyanidines or procyanidines, thearubigenin;
flavones and their glycosides, e.g. quercetin, quercitrin, rutin, taxifolin, myricetin, myricitrin;
other polyphenols, e.g. γ-oryzanol, caffeic add ester;
bitter isothiocyanates or substances derived therefrom such as thiocarbamate, thiourethane, glucosinolate, goitrin or propyl thiouracil (PROP) or phenylthiocarbamat (PTC);
terpenoid bitter principles, e.g. menthol, limonoids such as limonin or nomilin from citrus fruits, lupolones and humolones from hops, as well as iso-alpha acids derived therefrom, iridoids, secoiridoids, absinthin from wormwood, amarogentin from gentian;
pharmaceutical active ingredients, e.g. fluoroquinolone antibiotics, paracetamol (acetaminophen), aspirine, beta-lactam antibiotics, ambroxol, propyl thiouracil [PROP], omeprazole, guaifenesin, chloroquine;
denatonium benzoate or other denatonium salts;
sucralose octaacetate;
urea;
amino acids, e.g. leucine, isoleucine, valine, tryptophane, proline, histidine, tyrosine, lysine and phenylalanine;
peptides, in particular peptides with an amino acid from the group comprising leucine, isoleucine, valine, tryptophane, proline or phenylalanine at the N- or C-terminus
   as well as mixtures thereof.

Particularly preferred are substances, which may trigger a bitter taste in aqueous solvent systems in humans in a suitable amount alone and which, preferably, may at least activate one or more bitter receptor types and which are selected from the group of caffeine, theobromine, theophylline, salicin, sinigrin, arbutin, quinine, menthol, optionally galloylisedcatechins or epicatechins such as epigallocatechin, epigallocatechingallate, epicatechingallate, amarogentin, limonoides such as limonin or nomilin form citrus fruits, lupolone as well as iso-alpha-acids derived therefrom, amino acids (e.g. leucine, isoleucine, valine, tryptophane, proline, histidine, tyrosine, lysine or phenylalanine), and peptides (in particular peptides with an amino acid from the group of leucine, isoleucine, valine, tryptophane, proline or phenylalanine at the N- or C-terminus).

With the above-mentioned method bitter tasting compounds not identified so far, of course, can be identified.

### Test Substances

Since it is one object of the present method to identify candidate compounds which have bitter taste modulating characteristics, a complete list of potential test substances and compounds cannot be added. However, it is possible to indicate classes of compounds which form a basis for a search for suitable candidate compounds. For bitter taste masking substances, the groups of hydroxyflavones, hydroxybenzoic acid amides, hydroxydeoxybonzoines, hydroxyphenylalkanediones, 4-hydroxydihydrochalcons as well as vanillyl lignanes can be named.

### Measuring Method In Vitro

In the following experimental part it will be described in detail how to carry out the method according to the present invention. Therefore, only some general remarks will be made here.

The measurement of the intracellular pH value is performed spectrometically, preferably by using a fluorescent dye, which is suitable for the determination of the intracellular pH value between pH 6 and pH 8, for example 2′,7′-bis-(2-carboxypropyl)-5-(and-6-)-carboxyfluorescein (BCECF) and its esters and/or its salts, 8-hydroxypyren-1,3,6-trisulfonic acid (HPTS) and its salts and/or esters, carboxyfluorescein and its esters and/or its salts, 1,5-carboxy-seminaphto-rhodafluor (SNARF) and its esters and/or salts, or further fluorescent dyes described in Chem Rev. 2010 110(5):2709-2728; in particular, however, 1,5-carboxy-seminaphto-rhodafluor-acetoxymethylester (SNARE-1-AM). As an alternative dye, acridine orange as well as the measurement of C¹⁴ labelled amino pyrine is considered, which method is known as amino pyrine uptake (Ding, X.; Deng, H.; Wang, D.; Zhou, J.; Huang, Y.; Zhao, X.; Yu, X.; Wang, M.; Wang, F.; Ward, T.; Aikhionbare, F.; Yao, X., Phospho-regulated ACAP4-Ezrin interaction is essential for histamine-stimulated parietal cell secretion. J Biol Chem 2010, 285, 18769-80).

The method according to the present invention is performed on the basis of the change in the pH value in cultures of proton secreting gastric cells (preferably of type HGT-1) with or without test substance added. It is recommendable to maintain the experiments for a minimal time period in order to monitor the change in the pH value, i.e. to determine the pH value not before an equilibrium is reached. For the stimulation of the cells, a time period of 10 minutes is sufficient so that the duration of the experiment should be about 1 to about 30 minutes, preferably about 5 to about 20 minutes and, in particular about 8 to about 14 minutes.

The cell cultures are conventionally incubated along with the dye and then divided wherein to one half the well-known bitter tasting compound and to the other half the mixture of the same bitter tasting compound and one or more test substances is added. Typical amounts of a known bitter agonist are 50 to 150 μl, the test substances usually will be used in concentrations of about 0.1 to 3,000 μM. It turned out to be advantageous to use test substances in differing amounts, for example in concentrations of 0.1, 1, 10, 100 and 1,000 μM in order to exclude that a suitable candidate remains unrecognized, for example, since it has been used in a too small amount.

From the ratio of wavelength at excitation and emission of the fluorescent dye, a calibration curve can be established, based on which the pH value in the samples can be easily determined. For the calibration curve, the cells preferably will be treated with a potassium buffer having varying pH values of from 7.2 to 8.2 pH and 2 μM nigericin. Nigericin equilibrates the intracellular and extracellular pH value, so that the intracellular pH value can be defined. The intracellular H⁺ concentration then is derived from the intracellular pH value. The reduced amount of intracellular protons and the amount of released protons, respectively, will be calculated by log 2 transformation of the ratio of treated cells and untreated cells (control). The results so received then will be indicated as percent change compared to the untreated control cells (see Malte Rubach, Roman Lang, Elisabeth Seebach, Mark M. Somoza, Thomas Hofmann, Veronika Somoza; Mol Nutr Food Res. 2012, 56:325-35; Rubach, M.; Lang, R.; Hofmann, T.; Somoza, V., Ann N Y AcadSci 2008, 1126, 310-4; Rubach, M.; Lang, R.; Skupin, C.; Hofmann, T.; Somoza, V., J Agric Food Chem 2010, 58, 4153-61; Weiss, C.; Rubach, M.; Lang, R.; Seebach, E.; Blumberg, S.; Frank, 0.; Hofmann, T.; Somoza, V., J Agric Food Chem 2010, 58, 1976-85; Liszt, K. I.; Walker, J.; Somoza, V., J Agric Food Chem 2012; Walker, J.; Hell, J.; Liszt, K. I.; Dresel, M.; Pignitter, M.; Hofmann, T.; Somoza, V., J Agric Food Chem 2012, 60, 1405-12).

### Measuring Method In Vivo

The method according to the present invention may, in a further aspect of the present invention, also be performed in vivo. For example, the above described method may be performed by administering a known bitter tasting compound and/or a test substance to a test animal or person in encapsulated form such as a capsule or tablet. The formulation has to be swallowed and its ingredients have to be released in the stomach solely. If solid or liquid preparations are administered, oral taste receptors might be activated which also has an impact on mechanisms regulating gastric acid secretion. Thus, a direct comparison between the effects seen for proton-secreting gastric cells in culture and results obtained for a functioning stomach by applying the Heidelberg capsule systems requires avoiding the activation of oral taste receptors.

The gastric pH of the test animal or person then is measured over a defined time period, preferably by using a non-invasive measurement such as the Heidelberg Detection System (Heidelberg Medical Inc., USA). This system consists of a pH-sensitive capsule (called a Heidelberg capsule), with a length of 2 cm, that has to be swallowed and contains a miniature radio transmitter. This system allows the detection of the actual gastric pH of the volunteer over a specific time period.

The influence of the test substance on the proton secretion of gastric cells then can be measured as the gastric pH and the above calculations be performed in order to identify a suitable test substance.

### INDUSTRIAL APPLICABILITY

The method of the present invention is suitable in order to identify different taste modulating and bitter modulating as well as bitter tasting compounds, namely bitter taste masking agents and bitter taste enhancers, respectively. The area of application comprises both, the area of food/nutrition and the pharmaceutical area.

### Food/Nutrition

The food products to which bitter taste masking or bitter taste enhancing compounds, preferably however, bitter taste masking compounds, identified according to the present invention can be added are baked goods (e.g. bread, dry biscuits, cakes, other pastries), confectionery (e.g. chocolates, chocolate bar products, other bar products, fruit gums, hard and soft caramels, chewing gum), alcoholic or non-alcoholic drinks (e.g. coffee, tea, wine, wine-based drinks, beer, beer-based drinks, liqueurs, spirits, brandies, fruit-based soft drinks, isotonic drinks, soft drinks, nectars, fruit and vegetable juices, fruit or vegetable juice preparations), instant drinks (e.g. instant chocolate drinks, instant tea drinks, instant coffee drinks), meat products (e.g. ham, cured or uncured sausage preparations, spiced or marinated fresh or salted meat products), eggs or egg products (dried egg, egg white, egg yolk), cereal products (e.g. breakfast cereals, muesli bars, pre-fermented prepared rice products), dairy products (e.g. milk drinks, ice cream, yoghurt, kefir, cream cheese, soft cheese, hard cheese, dried milk powder, whey, butter, buttermilk), products made from soya protein or other soya bean fractions (e.g. soya milk and products made therefrom, preparations containing soya lecithin, fermented products such as tofu or tempe or products made therefrom), fruit preparations (e.g. jams, fruit sorbets, fruit sauces, fruit fillings), vegetable preparations (e.g. ketchup, sauces, dried vegetables, frozen vegetables, pre-fermented vegetables, preserved vegetables), snacks (e.g. baked or fried potato crisps or potato dough products, extruded products based on maize or peanuts), products based on fats and oils or emulsions thereof (e.g. mayonnaise, remoulade, dressings), other ready meals and soups (e.g. dried soups, instant soups, pre-fermented soups), spices, spice mixes and in particular seasonings, which are used in the snacks sector for example.

### Pharmaceutical Preparations

Pharmaceutical preparations, to which bitter tasting, bitter taste masking or bitter taste enhancing compounds, preferably however bitter masking compounds, can be added, are preparations containing already bitter tasting active pharmaceutical ingredients. A list of potentially naturally occurring and synthetic bitter pharmaceutical active compounds are published in Meyerhof, W.; Batram, C.; Kuhn, C.; Brockhoff, A.; Chudoba, E.; Bufe, B.; Appendino, G.; Behrens, M., The Molecular Receptive Ranges of Human TAS2R Bitter Taste Receptors. Chemical Senses 2010, 35, (2), 157-170, the database http://bitterdb.agri.huji.ac.il/bitterdb/ and Clark, A. A.; Liggett, S. B.; Munger, S. D., Extraoral bitter taste receptors as mediators of off-target drug effects. FASEB Journal 2012, 26, (12), 4827-4831. The following list is cited from the latter publication Bitter-tasting drugs and other bioactive compounds and their cognate human T2Rs according to Clark et al.:

| | | Responsive T2R |
|---|---|---|
| Drug | Action | isoforms |
| Acetaminophen | Analgesic | 39 |
| Aloin | Laxative | 31, 43 |
| Azathioprine | Immunosuppressive | 4, 10, 14, 46 |
| Carisoprodol | Muscle relaxant | 14, 46 |
| Chloramphenicol | Antibiotic | 1, 8, 10, 39, 43, 46 |
| Chloroquine | Antimalarial | 3, 7, 10, 39 |
| Colchicine | Gout | 4, 39, 46 |
| Cromoglicic acid | Mast cell stabilizer | 7, 20, 43 |
| Dapsone | Topical Antibacterial | 4, 10, 40 |
| Dextromethorphan | Antitussive | 1, 10 |
| Diphenhydramine | Antihistamine | 14, 40 |
| Diphenidol | Antiemetic | 1, 4, 7, 10, 13, 14, |
| | | 16, 20, 30, 31, 38, |
| | | 39, 40, 43, 46 |
| Erythromycin | Antibiotic | 10 |
| Famotidine | Gastric acid Inhibitor | 10, 31 |
| Flufenamic acid | Anti-inflammatory | 14 |
| Haloperidol | Antipsychotic | 10, 14 |
| Hydrocortisone | Glucocorticoid | 46 |
| Methimazole | Antithyroid | 38 |
| Noscapine | Antitussive | 14 |
| Orphenadrine | Antispasmodic | 14, 46 |
| Papaverine | Antispasmodic | 7, 10, 14, 31, 46 |
| Pirenzapine | Gastric acid Inhibitor | 9 |
| Propylthiouracil | Antithyroid | 38 |
| Procainamide | Antiarrhythmic | 9 |
| Ofloxacin | Antibiotic | 9 |
| Quinine | Antimalarial | 4, 7, 10, 14, 31, |
| | | 39, 40, 43, 46 |

Particularly preferred pharmaceutical preparations are preparations not subject to medical prescription, so called OTC (over the counter) preparations containing active pharmaceutical ingredients such as acetaminophen, acetylsalicylic acid or ibuprofen, dextromethorphan, hydrocortisone, vitamins (e.g. vitamin H, vitamins from the B-series such as vitamin B1, B2, B6, B12, niacin, panthotenic acid, preferably in the form of (effervescent) tablets or capsules), minerals (preferably in form of (effervescent) tablets or capsules) such as iron salts, zinc salts, selenium salts, products containing active pharmaceutical ingredients or extracts of buckhorn (e.g. cough syrup) or amber.

The industrial applicability of the method of the present invention can be extended to screening (e.g. rapid or high-throughput screening) of potential bitter-masking substances in order to identify those substances that effectively reduce the bitter-taste perception of a known bitter-tasting substance. The bitter-tasting substance may be a food or food additive/supplement ingredient, nutraceutical ingredient or a pharmaceutical compound or formulation. Furthermore, the method of the present invention can be used in pharmaceutical drug development programs, for instance to screen a selection of drug candidate molecules (e.g. small chemical entities) in order to identify those with a bitter taste profile, so that the drug development strategy can be adapted accordingly (for instance by excluding those molecules, or by designing modifications intended to reduce the bitter taste effects). Further, the present method may be of value for identifying/classifying the bitter taste of different plant extracts, for example extracts derived from the same plant but using different extraction methods. For example, extracts with a suitably low bitter taste profile then might be used as food additive or as pharmaceutical preparation.

It should be clear that this enumeration is only for illustrative purposes and will not limit the present invention.

### EXAMPLES

### Cell Culture

The human gastric tumor cell line HGT-1 was used for all cell culture experiments. This cell line has been obtained from Dr. C. Laboisse (Laboratory of Pathological Anatomy, Nantes, France). The cells were cultured under standard conditions at 37° C., 95% humidity, and 5% CO₂ in DMEM with 4 g/L glucose, 10% fetal bovine serum, 2% L-glutamine, and 1% penicillin/streptomycin. For the reverse transcription and the intracellular proton concentration assay, the cells were harvested using trypsin/EDTA. Cell viability has been determined using trypan blue staining for which the cells were seeded in a defined cell number in 35 mm dishes respectively in black 96-well plates.

### Example 1

### Identification of the Expression of Bitter Taste Receptors in HGT-1 Cells Using RT-qPCR

RNA of HGT-1 cells was isolated using the peqGold Total RNA Kit (Peqlab). RNA quantity and quality were checked spectrophotometrically. High Capacity cDNA Reverse Transcription Kit (Applied Biosystems) was used for cDNA synthesis following the manufacturer's protocol. Primers were designed using the primer designing tool of NCBI (using Primer 3 and Blast). Real-time PCR assays were performed on a StepOne plus (Applied Biosystems), using the Fast SYBR green master mix (Applied Biosystems). Cycling conditions were set as follows: 20 s/95° C. (activation), 3 s/95° C. (denaturation), 30 s/60° C. (annealing), 15 s/67° C. (elongation with fluorescence measurement). The PCR products were analysed by recording melting curves and determination of amplicon length on an agarose gel. qPCR data was analysed using the LinReg PCR software (free online software). This software can calculate the starting concentration (No) per sample, expressed in arbitrary fluorescence units. The calculated starting concentrations of the TAS2Rs were compared or normalized to the starting concentrations of the acetylcholine receptor (CHRM3), a receptor which is typically expressed in parietal cells on a functional level.

The following Table 1 represents the mRNA expression of the bitter taste receptors in HGT-1 cells normalized to the expression of the acetylcholine receptor (CHRM3). Data is shown as mean±standard deviation; n=3 (n, biological replicates), tr=3 (tr, technical replicates).

TABLE 1
mRNA of TAS2Rs is similarly or even
higher expressed as mRNA of CHRM3

| Receptor | Ratio to CHRM3 | SD |
|---|---|---|
| CHRM3 | 1.00 | 0.16 |
| TAS2R1 | 0.22 | 0.22 |
| TAS2R3 | 14.14 | 7.38 |
| TAS2R7 | 0.19 | 0.06 |
| TAS2R10 | 1.71 | 0.94 |
| TAS2R14 | 14.44 | 5.18 |
| TAS2R16 | 0.75 | 0.31 |
| TAS2R20 | 9.09 | 3.42 |
| TAS2R30 | 11.19 | 4.75 |
| TAS2R38 | 0.07 | 0.05 |
| TAS2R40 | 0.71 | 0.24 |
| TAS2R43 | 6.85 | 0.97 |
| TAS2R46 | 2.47 | 0.79 |
| TAS2R50 | 3.85 | 1.71 |

### Example 2

### Identification of Potential Bitter Substances Through Stimulation of Proton Secretion in HGT-1 Cells

For the measurement of the intracellular pH as indicator for proton secretion in HGT-1 cells, the pH-sensitive fluorescence day 1,5carboxy-seminaphto-rhodafluoracetoxymethylester (SNARF-1-AM) was used.

In a 96-well plate, a total of 100 000 HGT-1 cells were spread and allowed to settle for 24 h at 37° C., 95% humidity, and 5% CO₂. Cells were washed once with Krebs-Ringer-HEPES buffer (KRHB), and incubated with the fluorescence dye SNARF-1-AM at a concentration of 3 μM for 30 min. Afterward, cells were washed twice with KRHB and treated with 100 μL of caffeine, and theobromine in different concentrations diluted in phenol red free media for ten minutes. As positive control, the cells were treated with 1 mM histamine.

Fluorescence was analyzed at an excitation of 488 nm, whereas emission wavelengths were recorded at 580 nm and 640 nm on an Infinite 200 Pro plate reader. The ratio of the fluorescence intensities from those two emission wavelengths allows an accurate determination of pH when plotted on a calibration curve. For each experiment, a calibration curve was generated by staining the cells in potassium buffer solutions of varying pH values, ranging from 7.2 to 8.2 adjusted with NaOH, using a pH-meter pH 211 (HANNA Instruments), in the presence of 2 μM nigericin to equilibrate intracellular pH and extracellular pH. The potassium buffer calibration solutions for the intracellular pH measurement consisted of 20 mM NaCl, 110 mM KCl, 1 mM CaCl₂, 1 mM MgSO₄, 18 mM D-glucose, and 20 mM HEPES. The pH calibration was fit to a linear regression. Intracellular proton concentration was calculated from the pH. The reduced amount of protons in the cell was calculated by log 2 transformation of the ratio between treated and untreated cells (control). The presented data is given as percent variation in comparison to untreated cells.

The following Table 2A shows the proton secretion in HGT-1 cells after 10 min. stimulation with histamine (1 mM, positive control) or caffeine in different concentrations in comparison to untreated cells (control). Data is displayed as mean±SEM, n=5, tr=6; Statistic: one-way Anova with post-hoc Test after Dunn's. Significant differences (p<0.05) are indicated by letters.

TABLE 2A
Proton secretion after treatment with control substance
histamine and the bitter substance caffeine

| Test compound | T/C [%] | SEM |
|---|---|---|
| Control | 1.09 | 5.14 a |
| Histamine 1 mM | 36.66 | 4.93 b |
| Caffeine 0.3 μM | 25.49 | 5.02 a, b |
| Caffeine 3 μM | 14.74 | 3.60 a, b |
| Caffeine 30 μM | 23.43 | 3.91 a, b |
| Caffeine 300 μM | 17.07 | 3.54 a, b |
| Caffeine 3000 μM | 40.97 | 2.72 b |

The following Table 2B shows the proton secretion in HGT-1 cells after 10 min. stimulation with histamine (1 mM, positive control) or theobromine in different concentrations in comparison to untreated cells (control). Data is displayed as mean±SEM, n=4, tr=6; Statistic: one-way Anova with post-hoc Test after Dunn's. Significant differences (p<0.05) are indicated by letters.

TABLE 2B
Proton secretion after treatment with control substance
histamine and the bitter substance theobromine

| Test compound | T/C [%] | SEM |
|---|---|---|
| Control | −3.25 | −5.64 a |
| Histamine 1 mM | 45.70 | −5.87 b |
| Theobromine 0.03 μM | 3.71 | −7.99 c, a |
| Theobromine 0.3 μM | 17.71 | −9.06 c, a |
| Theobromine 3 μM | 43.16 | −11.6 b, c |
| Theobromine 30 μM | 44.88 | −7.4 b |
| Theobromine 300 μM | 57.2 | −11.18 b |

### Example 3

### Identification of Bitter-Masking Compounds by Measurement of the Reduction of Bitter Substances Induced Proton Secretion in HGT-1 Cells

For the measurement of the intracellular pH as indicator for proton secretion in HGT-1 cells, the pH-sensitive fluorescence day 1,5carboxy-seminaphto-rhodafluoracetoxymethylester (SNARF-1-AM) was used.

In a 96-well plate, a total of 100,000 HGT-1 cells were spread and allowed to settle for 24 h at 37° C., 95% humidity, and 5% CO₂. Cells were washed once with KRHB and incubated with the fluorescence dye SNARF-1-AM at a concentration of 3 μM for 30 min. Afterward, cells were washed twice with KRHB and treated with 100 μL of the bitter substances, 3 mM caffeine respectively 0.3 mM theobromine alone or in combination with the bitter masking compounds homoeriodictyol (HED) or eriodictyol or matairesinol or lariciresinol in different concentrations diluted in phenol red free media for ten minutes. As positive control, the cells were treated with 1 mM histamine.

Fluorescence was analyzed at an excitation of 488 nm and emission wavelengths were recorded at 580 nm and 640 nm on an Infinite 200 Pro plate reader. The ratio of the fluorescence intensities from those two emission wavelengths allows an accurate determination of pH when plotted on a calibration curve.

For each experiment, a calibration curve was generated by staining the cells in potassium buffer solutions of varying pH values, ranging from 7.2 to 8.2 adjusted with NaOH using a pH-meter pH 211 (HANNA Instruments), in the presence of 2 μM nigericin to equilibrate intracellular pH and extracellular pH. The potassium buffer calibration solutions for the intracellular pH measurement consisted of 20 mM NaCl, 110 mM KCl, 1 mM CaCl2, 1 mM MgSO4, 18 mM D-glucose, and 20 mM HEPES. The pH calibration was fit to a linear regression curve. Intracellular proton concentration was calculated from the pH. The reduced amount of protons in the cell was calculated by log 2 transformation of the ratio between treated and untreated cells (control). The presented data is given as percent variation in comparison to untreated cells.

The following Table 3A shows the percent increase in proton secretion in HGT-1 cells in comparison to untreated cells (control) after 10 min. stimulation with 3 mM caffeine or 3 mM caffeine in combination with homoeriodictyol (HED) in different concentrations. Data is displayed as mean±SEM, n=4, tr=6; Statistic: one-way Anova with post-hoc Test after Dunn's. Significant differences (p<0.05) are indicated by letters.

TABLE 3A
Percent increase of caffeine alone or in combination with
the bitter-masking compound homoeriodictyol on proton secretion
in HGT-1 cells compared to non-treated controls (=0).

| Test compound | T/C [%] | SEM |
|---|---|---|
| 3 mM Caffeine | 54.79 | 5.61 a |
| 3 mM Caffeine + 0.03 mM HED | 37.99 | 6.70 a |
| 3 mM Caffeine + 0.3 mM HED | 20.18 | 5.99 b |

The following Table 3B shows the percent increase in proton secretion in HGT-1 cells in comparison to untreated cells (control) after 10 min. stimulation with 3 mM caffeine or 3 mM caffeine in combination with eriodictyol in different concentrations. Data is displayed as mean±SEM, n=4, tr=6; Statistic: one-way Anova with post-hoc Test after Dunn's. Significant differences (p<0.05) are indicated by letters.

TABLE 3B
Percent increase of caffeine alone or in combination with
the bitter-masking compound eriodictyol on proton secretion
in HGT-1 cells compared to non-treated controls (=0).

| Test compound | T/C [%] | SEM |
|---|---|---|
| 3 mM Caffeine | 54.05 | 4.37 a |
| 3 mM Caffeine + 0.03 mM Eriodictyol | 29.69 | 8.82 a |
| 3 mM Caffeine + 0.3 mM Eriodictyol | −33.96 | 7.55 b |

The following Table 3C shows the percent increase in proton secretion in HGT-1 cells in comparison to untreated cells (control) after 10 min. stimulation with 3 mM caffeine or 3 mM caffeine in combination with matairesinol in different concentrations. Data is displayed as mean±SEM, n=4, tr=6; Statistic: one-way Anova with post-hoc Test after Dunn's. Significant differences (p<0.05) are indicated by letters.

TABLE 3C
Percent increase of caffeine alone or in combination with
the bitter-masking compound matairesinol on proton secretion
in HGT-1 cells compared to non-treated controls (=0).

| Test compound | T/C [%] | SEM |
|---|---|---|
| 3 mM Caffeine | 38.75 | 2.63 a |
| 3 mM Caffeine + 0.03 mM Matairesinol | 14.39 | 3.55 b |
| 3 mM Caffeine + 0.3 mM Matairesinol | −20.43 | 4.82 c |

The following Table 3D shows the percent increase in proton secretion in HGT-1 cells in comparison to untreated cells (control) after 10 min. stimulation with 3 mM caffeine or 3 mM caffeine in combination with lariciresinol in different concentrations. Data is displayed as mean±SEM, n=5, tr=6; Statistic: one-way Anova with post-hoc Test after Dunn's. Significant differences (p<0.05) are indicated by letters.

TABLE 3D
Percent increase of caffeine alone or in combination with
the bitter-masking compound lariciresinol on proton secretion
in HGT-1 cells compared to non-treated controls (=0).

| Test compound | T/C [%] | SEM |
|---|---|---|
| 3 mM Caffeine | 52.41 | 4.16 a |
| 3 mM Caffeine + 0.03 mM Lariciresinol | 46.40 | 3.98 a |
| 3 mM Caffeine + 0.3 mM Lariciresinol | 21.57 | 3.78 b |

The following Table 3E shows the percent increase in proton secretion in HGT-1 cells in comparison to untreated cells (control) after 10 min. stimulation with 0.3 mM theobromine or 0.3 mM theobromine in combination with homoeriodictyol (HED) in different concentrations. Data is displayed as mean±SEM, n=3, tr=6; Statistic: one-way Anova with post-hoc Test after Dunn's. Significant differences (p<0.05) are indicated by letters.

TABLE 3E
Percent increase of theobromine alone or in combination with
the bitter-masking compound homoeriodictyol (HED) on proton secretion
in HGT-1 cells compared to non-treated controls (=0).

| Test compound | T/C [%] | SEM |
|---|---|---|
| 0.3 mM Theobromine | 20.92 | 3.37 a |
| 0.3 mM Theobromine + 0.003 mM HED | 21.56 | 2.33 a |
| 0.3 mM Theobromine + 0.03 mM HED | 13.79 | 3.50 a |
| 0.3 mM Theobromine + 0.3 mM HED | −16.92 | 3.27 b |

The following Table 3F shows the percent increase in proton secretion in HGT-1 cells in comparison to untreated cells (control) after 10 min. stimulation with 0.3 mM theobromine or 0.3 mM theobromine in combination with eriodictyol in different concentrations. Data is displayed as mean±SEM, n=3, tr=6; Statistic: one-way Anova with post-hoc Test after Dunn's. Significant differences (p<0.05) are indicated by letters.

TABLE 3F
Percent increase of theobromine alone or in combination with
the bitter-masking compound eriodictyol on proton secretion
in HGT-1 cells compared to non-treated controls (=0).

| Test compound | T/C [%] | SEM |
|---|---|---|
| 0.3 mM Theobromine | 47.64 | 5.28 a |
| 0.3 mM Theobromine + 0.03 mM Eriodictyol | 23.95 | 4.17 b |
| 0.3 mM Theobromine + 0.3 mM Eriodictyol | −42.86 | 9.73 c |

The following Table 3G shows the percent increase in proton secretion in HGT-1 cells in comparison to untreated cells (control) after 10 min. stimulation with 0.3 mM theobromine or 0.3 mM theobromine in combination with matairesinol in different concentrations. Data is displayed as mean±SEM, n=3, tr=6; Statistic: one-way Anova with post-hoc Test after Dunn's. Significant differences (p<0.05) are indicated by letters.

TABLE 3G
Percent increase of theobromine alone or in combination with
the bitter-masking compound matairesinol on proton secretion
in HGT-1 cells compared to non-treated controls (=0).

| Test compound | T/C [%] | SEM |
|---|---|---|
| 0.3 mM Theobromine | 34.12 | 2.86 a |
| 0.3 mM Theobromine + 0.03 mM Matairesinol | 23.41 | 2.56 b |
| 0.3 mM Theobromine + 0.3 mM Matairesinol | −18.25 | 3.39 c |

The following Table 3H shows the percent increase in proton secretion in HGT-1 cells in comparison to untreated cells (control) after 10 min. stimulation with 0.3 mM theobromine or 0.3 mM theobromine in combination with lariciresinol in different concentrations. Data is displayed as mean±SEM, n=3, tr=6; Statistic: one-way Anova with post-hoc Test after Dunn's. Significant differences (p<0.05) are indicated by letters.

TABLE 3H
Percent increase of theobromine alone or in combination with
the bitter-masking compound lariciresinol on proton secretion
in HGT-1 cells compared to non-treated controls (=0).

| Test compound | T/C [%] | SEM |
|---|---|---|
| 0.3 mM Theobromine | 30.81 | 3.44 a |
| 0.3 mM Theobromine + 0.03 mM Lariciresinol | 31.73 | 5.80 a |
| 0.3 mM Theobromine + 0.3 mM Lariciresinol | 17.55 | 3.67 a |
| | | (t-test p < 0.05) |

These results clearly demonstrate that the HGT-1 cell line is suitable for identifying bitter-masking compounds.

### Example 4

### Sensoric Assessment: Bitter-Reduction of a Bitter Tasting Solution

The results in the following Table 4 were collected as described in EP 2,517,574: For quantification of the reduction or masking of the bitter impression of a sample, the bitterness of a bitter substance in defined concentrations was compared by a sensorically-trained expert panel using a sample solution which included the same concentration of the same bitter substance in addition with a potential bitter masking or bitter reducing compound in the concentration given in Table 4. For categorization of the bitter impression, a scale from 1 [not bitter] to 10 [extremely bitter] was used. For calculation of the percent reduction of bitter taste, the mean values of the rating of the expert panel for the solution of the bitter substance alone and the solution of the bitter substance in combination with the tested potential masking substance were compared. Significances were calculated using a paired, two sided student t-test and indicated by the p-values in Table 4.

TABLE 4
Sensoric assessment

| | Bitterness | | Bitterness | |
|---|---|---|---|---|
| | Bitter compound | | Bitter compound + | % Reduction of the |
| Bitter compound | alone | Test compound | Test substance | Bitter impression |
| Caffeine | 6.3 ± 1.4 | Homoeriodictyol | 4.5 ± 1.4 | 28% |
| (500 ppm) | | (100 ppm) | | (p < 0.001) |
| Theobromine | 4.3 ± 1.8 | Homoeriodictyol | 3.7 ± 1.3 | 12.5% |
| (300 ppm) | | (100 ppm) | | |
| Caffeine | 6.7 ± 1.1 | Eriodictyol | 3.5 ± 0.7 | 47% |
| (500 ppm) | | (100 ppm) | | (p < 0.0005) |
| Theobromine | 3.8 ± 1.8 | Eriodictyol | 2.4 ± 1.2 | 36% |
| (300 ppm) | | (100 ppm) | | (p < 0.01) |
| Caffeine | 4.1 ± 0.9 | Matairesinol | 2.7 ± 1.77 | 35.5% |
| (500 ppm) | | (25 ppm) | | (p < 0.05) |
| Theobromine | 4.5 ± 2.01 | Matairesinol | 3.4 ± 1.49 | 24.7% |
| (300 ppm) | | (25 ppm) | | |
| Caffeine | 4.7 ± 1.11 | Lariciresinol | 2.9 ± 1.41 | 38.6% |
| (500 ppm) | | (25 ppm) | | (p < 0.05) |
| Theobromine | 4.2 ± 1.65 | Lariciresinol | 3.7 ± 2.24 | 10.7% |
| (300 ppm) | | (25 ppm) | | |

### Example 5

### Reduction of the Expression of TAS2R10 by Means of a siRNA Knockdown Targeted Against TAS2R10 Reduces the Effect of Caffeine on Proton Secretion

The HiPerFect transfection reagent (Qiagen) was used to transfect small interfering RNA (siRNA), targeting specifically human TAS2R10 (5′-GACACAGUCUGGGAUCUCA-3′; Sigma-Aldrich) into HGT-1 cells for the specific reduction of TAS2R10 expression. Cells were grown to 50% confluence in serum containing DMEM-media and incubation for 48 h was started by addition of serum-free media containing siRNA targeted against TAS2R10 (final siRNA concentration 1 nM) and HiPerFect transfection reagent (1 μL/6 pmol siRNA). Unrelated non-silencing siRNA (Qiagen) was used as negative control and siRNA targeted against Mn/Hs_MAPK1 which is known to efficiently knocks down human MAPK1 (Qiagen) was used as positive control. A mock transfection, in which only the HiPerFect transfection reagent was tested, was also carried out to exclude the effects of the transfection reagent itself. The knockdown efficiency was controlled by measuring mRNA expression of TAS2R10 using RT-qPCR as explained in example 1. Transfection of HGT-1 cells with siRNA directed against TAS2R10 decreased TAS2R10 mRNA levels by 29±8% compared to untreated cells. Mock transfection and non-silencing siRNA transfection showed no influence on mRNA expression. Transfection of HGT-1 cells with siRNA targeted against Hs_MAPK1, the positive control, decreased the MAPK1 mRNA levels by 58±4% compared to not transfected cells. The effect of 3 mM caffeine in non-transfected, mock transfected and siRNA “knockdown” HGT-1 cells on proton secretion was measured over a time course of 30 minutes with an interval of 5 minutes using the ph-sensitive fluorescence dye SNARF-1-AM as described in example 2.

Table 5 shows the percent effect of the proton secretion in HGT-1 cells in comparison to untreated cells after 10 min treatment with 3 mM caffeine on mock transfected and siRNA “knockdown” HGT-1 cells in comparison to not transfected cells. Statistics: two-way ANOVA with Student-Newman-Keuls post-hoc test, data shown as mean±SEM, n=3, tr=6.

TABLE 5
Percent effect of caffeine on proton secretion in TAS2R10
“knockdown” HGT-1 cells (data shown as mean ±
SEM, n = 3 biological replicates with 6 technical replicates each.

| Test compound, cell treatment | T/C [%] | SEM |
|---|---|---|
| Control, not transfected cells | 0.61 | 2.75 a |
| Control, mock transfected cells | 8.33 | 4.57 a |
| Control, siRNA TAS2R10 ,,knockdown” cells | 2.51 | 5.59 a |
| 3 mM Caffeine, not transfected cells | 48.80 | 5.61 b |
| 3 mM Caffeine, mock transfected cells | 52.80 | 4.99 b |
| 3 mM Caffeine, siRNA TAS2R10 ,,knockdown” cells | 28.58 | 8.30 c |

1. An in vitro method for the identification of bitter tasting compounds and bitter taste modulating compounds, the in vitro method comprising contacting test substances with isolated gastric cells or gastric tumor cells and determining the resulting change in the intracellular pH value and/or cellular proton secretion.

2. The method of claim 1, where the gastric cells or gastric tumor cells are proton secreting cells.

3. The method of claim 1, wherein bitter tasting compounds, bitter taste masking or bitter taste enhancing compounds are identified.

4. The method of claim 2, wherein the proton secreting gastric cell lines are selected from HGT-1, MKN-45, or AGS.

5. The method of claim 1, wherein bitter taste masking compounds are identified fulfilling the condition:
T/C(known bitter tasting compound)−T/C(known bitter tasting compound+test substance)>0
where T/C is the value of the proton secretion in percent, and where the relative difference between both T/C values is at least 10% of the higher value.

6. The method of claim 1, wherein bitter taste enhancing compounds are identified fulfilling the condition:
T/C(known bitter tasting compound)−T/C(known bitter tasting compound+test substance)<0
where T/C is the value of the proton secretion in percent, and where the relative difference between both T/C values is at least 10% of the higher value.

7. The method of claim 1, wherein bitter tasting compounds are identified fulfilling the condition:
T/C(control)−T/C(test substance)<0
where T/C is the value of the proton secretion in percent, and where the relative difference between both T/C values is at least 10% of the higher value.

8. The method of claim 1, where known bitter tasting compounds are used which are selected from the group of compounds capable of activating one or more bitter receptors, e.g. of type TAS2R1, TAS2R3, TAS2R7, TAS2R10, TAS2R14, TAS2R16, TAS2R20, TAS2R30, TAS2R38, TAS2R40, TAS2R43, TAS2R46 and TAS2R50.

9. The method of claim 8, where the bitter agonists are selected from xanthine alkaloids, alkaloids, phenolic glycosides, flavonoid glycosides, hydrolysable tannins and non-hydrolysable tannins, other polyphenols, bitter isothiocyanates or derived substances; terpenoid bitter principles; pharmaceutical active ingredients;
denatonium benzoate or other denatonium salts; sucralose octaacetate; urea; amino acids and peptides as well as mixtures thereof.

10. The method of claim 1, wherein the change in the intracellular pH value and/or the proton secretion of the cells is measured spectrometrically using a fluorescent dye.

11. The method of claim 1, wherein the change in the intracellular pH value and/or the proton secretion of the cells is measured over a time period of 1 to 30 min.

12. The method of claim 1, wherein the potential bitter tasting compounds are used in an amount of 50 to 150 μl.

13. The method of claim 1, wherein the test substances are used in an amount of 0.1 to 3,000 μM.

14. The method of claim 1, wherein for the identification of bitter taste modulating compounds the following steps are performed:
(a) a uniform culture of a cell system is provided selected from the group comprising isolated gastric cells or gastric tumor cells which is divided in two samples,
(b) to the first sample, a known bitter tasting compound is added,
(c) to the second sample, the same bitter tasting compound from step (b) and at least one test substance is added, which test substance can be one or more bitter taste modulating compounds,
(d) measuring the change in the intracellular pH value and/or the proton secretion of the cells during the course of the experiment,
(e) after completing the experiment, the difference between the change in the intracellular pH value and/or the proton secretion of the cells of the first and the second sample is calculated,
(f) those test substances are selected, where the difference is positive or negative and the relative difference between both values is at least 10% of the higher value, and
(g) optionally, subjecting the so identified test substances to a sensory evaluation.

15. The method of claim 1, where a bitter tasting compound is identified by performing the following steps:
(a) a uniform culture of a cell system is provided selected from the group comprising isolated gastric cells or gastric tumor cell lines, which is divided in two samples,
(b) to the first sample, a control solution without test substance is added,
(c) to the second sample, a test substance is added,
(d) the change in the intracellular pH value and/or proton secretion of the cells is measured during the course of the experiment,
(e) after completing the experiment, the difference between the change in the intracellular pH value and/or proton secretion of the cells of the first and the second sample is calculated,
(f) those test substances are selected where the difference is negative and where the relative difference between both values is at least 10% of the higher value,
(g) and, optionally, the so identified test substances subsequently are subjected to a sensory evaluation for the determination of the bitter effect.


SEQUENCE LISTING (1 sequence)

SEQ ID NO: 1 - RNA, 19 nt, artificial
  small interfering RNA (siRNA) designed specifically for human TAS2R10
gacacagucugggaucuca